(19) United States
(12) Patent Application Publication
(10) Pub. No.: US 2013/0338183 A1
(43) Pub. Date: Dec. 19, 2013

(54) COMPOSITIONS AND METHODS FOR MOBILIZING STEM CELLS

(71) Applicants: Zhaoli Sun, Perry Hall, MD (US); Takehiro Okabayashi, Baltimore, MD (US)

(72) Inventors: Zhaoli Sun, Perry Hall, MD (US); Takehiro Okabayashi, Baltimore, MD (US)

(73) Assignee: The Johns Hopkins University, Baltimore, MD (US)

(21) Appl. No.: 13/992,272

(22) PCT Filed: Dec. 7, 2011
(86) PCT No.: PCT/US2011/063727

§ 371 Date: Aug. 29, 2013

Related U.S. Application Data
(60) Provisional application No. 61/420,351, filed on Dec. 7, 2010.

(51) Int. Cl. A61K 31/436; A61K 31/395

(52) U.S. Cl. CPC A61K 31/436; A61K 31/395
USPC 514/291

(57) ABSTRACT

The present invention relates to the field of hematopoietic stem cells. More specifically, the present invention provides methods and composition useful for peripheral blood stem cell mobilization. In one embodiment, a method of treating an organ transplant recipient comprises administering to the recipient a low dose of Tacrolimus in an amount sufficient to mobilize stem cells to the peripheral blood of the recipient. In a specific embodiment, the low dose of Tacrolimus is in the range of about 0.05 mg/kg to about 0.5 mg/kg.

### CROSS-REFERENCE TO RELATED APPLICATIONS

This application claims the benefit of U.S. Provisional Application No. 61/420,351, filed Dec. 7, 2010; which is incorporated herein by reference in its entirety.

### FIELD OF THE INVENTION

The present invention relates to the field of hematopoietic stem cells. More specifically, the present invention relates to peripheral blood stem cell mobilization.

### BACKGROUND OF THE INVENTION

Peripheral blood stem cell mobilization is important as a source of hematopoietic stem cells for transplantation. Experimental studies and early phase clinical trials suggest that transplantation of blood-derived or bone marrow-derived stem cells may improve liver, kidney, cardiac and neuronal regeneration after injury. Bone marrow derived stem cells have the potential to improve the organ function after rejection through repairing the damaged tissues. Therefore, mobilization of bone marrow stem cells has broad clinical application.

### SUMMARY OF THE INVENTION

The present invention is based, at least in part, on the discovery that a low dose of the immunosuppressive drug Tacrolimus (FK-506) induces mobilization of bone marrow stem cells. Peripheral blood stem cell mobilization is important as a source of hematopoietic stem cells for transplantation. Mobilization of stem cells can promote the repair and regeneration of rejecting allografts after transplantation and eventually the allograft becomes recipient itself. Further, mobilization of stem cells can promote the repair and regeneration of damaged tissue/organs.

Side effects of Tacrolimus can be severe and include infection, cardiac damage, hypertension, blurred vision, liver and kidney problems, and even cancer. The risk appears to be related to the intensity and duration of treatment. A low dose of Tacrolimus is expected to result in minimal side effects. Therefore, there is no major obstacle to apply the present invention in clinical trials.

Accordingly, in one aspect, the present invention provides compositions and methods useful for mobilizing stem cells. In one embodiment, a method of treating an organ transplant recipient comprises administering to the recipient a low dose of Tacrolimus in an amount sufficient to mobilize stem cells to the peripheral blood of the recipient. In a specific embodiment, the low dose of Tacrolimus is in the range of about 0.05 mg/kg to about 0.5 mg/kg. In another specific embodiment, the low dose of Tacrolimus results in a blood concentration range of about 2 ng/ml to about 20 ng/ml. In an alternative embodiment, the low dose of Tacrolimus is about 0.05 mg/kg to about 0.1 mg/kg.

In particular embodiments, the organ is selected from the group consisting of liver, kidney, skin, heart, lung, intestine, and pancreas. In a specific embodiment, the organ is liver. In an alternative embodiment, the organ is kidney. In a further embodiment, the organ is skin.

In other embodiments, the treatment methods of the present invention further comprise administering a second agent to mobilize stem cells to the peripheral blood. The stem cell mobilizer can be selected from the group consisting of AMD3100, AMD3465, TG-0054, G-CSF, GM-CSF, SDF-1, and SCF. In a specific embodiment, the stem cell mobilizer is a CXCR4 antagonist. In a more specific embodiment, the stem cell mobilizer is AMD3100.

In another embodiment, the present invention provides a method of treating a liver transplant recipient comprising administering Tacrolimus to the recipient in an amount ranging from about 0.05 mg/kg to about 0.5 mg/kg. In yet another embodiment, a method of treating a kidney transplant recipient comprises administering Tacrolimus to the recipient in an amount ranging from about 0.05 mg/kg to about 0.5 mg/kg. In a further embodiment, a method of treating a skin transplant recipient comprises administering Tacrolimus to the recipient in an amount ranging from about 0.05 mg/kg to about 0.5 mg/kg. In an alternative embodiment, a method of treating a patient diagnosed with ischemic injury comprises administering Tacrolimus to the patient in an amount ranging from about 0.05 mg/kg to about 0.5 mg/kg. In another embodiment, the present invention provides a method of treating a composite tissue transplant recipient comprising administering Tacrolimus to the recipient an amount ranging from about 0.05 mg/kg to about 0.5 mg/kg.

In another specific embodiment, a method of treating an organ transplant recipient comprises administering a low dose of Tacrolimus to the recipient in an amount sufficient to mobilize CD34⁺ and/or CD133⁺ stem cells to the peripheral blood. In a more specific embodiment, the low dose of Tacrolimus is about 0.05 mg. to about 0.5 mg.

A method of treating an organ transplant recipient may comprise administering Tacrolimus at a dose of about 0.05 mg/kg to about 0.5 mg/kg to the recipient, wherein the dosage range is sufficient to mobilize CD34⁺ and/or CD133⁺ stem cells to the peripheral blood. In a more specific embodiment, a method of treating an organ transplant recipient comprising administering Tacrolimus at a dose of about 0.05 mg/kg to about 0.075 mg/kg to the recipient, wherein the dosage range is sufficient to mobilize CD34⁺ and/or CD133⁺ stem cells to the peripheral blood.

### BRIEF DESCRIPTION OF THE FIGURES

FIG. 1 is a graph showing the mobilization of bone marrow stem cells by low dose Tacrolimus (FK-506). FIG. 1A shows the absolute number of Lin-CD133+ cells in peripheral blood (per μl) following treatment with saline, 0.05 mg/kg FK-506, 0.1 mg/kg FK-506, or 1.0 mg/kg FK-506. FIG. 1B shows the percentage of Lin-CD133+ cells in spleen following treatment with saline, 0.05 mg/kg FK-506, 0.1 mg/kg FK-506, or 1.0 mg/kg FK-506.

### DETAILED DESCRIPTION OF THE INVENTION

The present invention is based, in part, on the discovery that the administration of a low dose of Tacrolimus can be used to mobilize stem cells to the peripheral blood. In particular embodiments, a low dose of Tacrolimus can be used to treat organ transplant recipients. The treatment regimen promotes allograft survival and induces long-term allograft acceptance. The treatment regimen can be applied to any type of organ transplant including liver, kidney, skin, heart, lung, intestine, and pancreas. The treatment regimen can also be applied to composite tissue transplantation. The composite tissue can be hand, face, or any other anatomical part.

The present invention consists of a novel strategy to mobilize recipient stem cells which can promote the repair and regeneration of rejecting allografts after transplantation and eventually the allograft becomes recipient itself. This allows minimal immunosuppression and rapid weaning. For patients, this translates into improved survival and elimination of immunosuppression related complications, such as infections and malignancy.

Although much of the present disclosure is made in the context of organ transplantation, it should be recognized that the treatment regimens are broadly applicable, as noted above, and should not be construed as limited to organ transplantation. In particular embodiments, the treatment regimen can be utilized for toxic liver injury such as acetaminophen or fulminent hepatitis. In general, however, the present invention is useful in the treatment of patients with ischemic injury and/or shock.

Moreover, the treatment regimen of the present invention likely recruits regulatory T-cells to the organ transplant site. Because regulatory T cells are involved in controlling autoimmune diseases including, but not limited to, type 1 diabetes, experimental autoimmune encephalomyelitis, and inflammatory bowel disease, the mobilization of stem cells (e.g., with a combination of AMD3100 and Tacrolimus) may have broader clinical applications rather than transplantation. In particular embodiments, therefore, the stem cell mobilizers and immunosuppressive agents can be used to treat autoimmune disease.

### I. Definitions

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into stem cells that are committed to three, two or one hematopoietic differentiation pathway. None of these stem cells are morphologically distinguishable, however.

The term “immunosuppressive agent” refers to an agent that inhibits, slows or reverses the activity of the immune system. Immunosuppressive agents act by suppressing the function of responding immune cells (including, for example, T cells), directly (e.g., by acting on the immune cell) or indirectly (by acting on other mediating cells). Immunosuppressive agents can be given to a subject to prevent the subject's immune system from mounting an immune response after an organ transplant or for treating a disease that is caused by an overactive immune system.

The terms “stem cells” and “hematopoietic stem cells” are used interchangeably herein. Stem cells are distinguished from other cell types by two important characteristics. First, stem cells are unspecialized cells capable of renewing themselves through cell division, sometimes after long periods of inactivity. Second, under certain physiologic or experimental conditions, stem cells can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

The term “stem cells” can refer to multipotent stem cells that are capable of differentiating into all blood cells including erythrocytes, leukocytes and platelets. For instance, the “hematopoietic stem cells” or “stem cells” as used in the invention are contained not only in bone marrow but also in umbilical cord blood derived cells.

A “stem cell mobilizer,” “mobilizer of hematopoietic stem cells or progenitor cells” or “mobilize,” (used interchangeably), as described herein, refers to any compound, whether it is a small organic molecule, synthetic or naturally derived, or a polypeptide, such as a growth factor or colony stimulating factor or an active fragment or mimic thereof, a nucleic acid, a carbohydrate, an antibody, or any other agent that acts to enhance the migration of stem cells from the bone marrow into the peripheral blood. A stem cell mobilizer may increase the number of hematopoietic stem cells or hematopoietic progenitor/precursor cells in the peripheral blood, thus allowing for a more accessible source of stem cells for use in transplantation. In particular embodiments, a stem cell mobilizer refers to any agent that mobilizes CD34⁺ and/or CD133⁺ stem cells.

A “patient,” “subject,” “host,” or “transplant recipient” to be treated by the present methods refers to either a human or non-human animal, such as primates, mammals, and vertebrates.

“Tacrolimus”, “FK-506” or “Fujimycin” (used interchangeably throughout) is an immunosuppressive agent that is mainly used after allogeneic organ transplant to reduce the activity of the patient's immune system and so lower the risk of organ rejection. It reduces interleukin-2 (IL-2) production by T-cells. It is also used in a topical preparation in the treatment of severe atopic dermatitis (eczema), severe refractory uveitis after bone marrow transplants, and the skin condition vitiligo. It is a 23-membered macrolide lactone discovered in 1984 from the fermentation broth of a Japanese soil sample that contained the bacteria Streptomyces tsukubaensis. The drug is sold under the trade names Prograf® given twice daily, Advagraf® a sustained release formulation allowing once daily dosing, and Protopic® the topical formulation.

As used herein, the terms “treatment,” “treating,” “treat” and the like, refer to obtaining a desired pharmacologic, and/or physiologic effect. The terms are also used in the context of the administration of a “therapeutically effective amount” of an agent, e.g., FK-506. The effect may be prophylactic in terms of completely or partially preventing a particular outcome, disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of treating organ transplant recipients. More particularly, treatment of an organ transplant recipient includes (a) achieving clinical tolerance; (b) promoting the repair and regeneration of rejecting allografts; (c) repopulating allografts with recipient-derived cells; (d) inducing long-term allograft acceptance without side effects; (e) reducing or eliminating immunosuppression related complications such as infections.

### II. Pharmaceutical Compositions And Administration

Accordingly, a pharmaceutical composition of the present invention may comprise an effective amount of a low dose of Tacrolimus. In such embodiments, Tacrolimus has characteristics of both a stem cell mobilizer and an immunosuppressive agent. As used herein, the term “effective,” means adequate to accomplish a desired, expected, or intended result. More particularly, an “effective amount” or a “therapeutically effective amount” is used interchangeably and refers to an amount of Tacrolimus, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat organ transplant recipients, patients with ischemic injury and/or shock, and/or autoimmune diseases. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The phrase “low dose” or “low dose amount” of Tacrolimus in the context of an effective amount to mobilize stem cells refers to the use of a particular amount of Tacrolimus that is lower than typically used for immunosuppression. In certain embodiments, the low dose is about 1/10 of the amount used for immunosuppression. In other embodiments, the low dose of is about ½, about ⅓, about ¼, about ⅕, about ⅙, about 1/7, about ⅛, or about 1/9 of the amount used for immunosuppression. In further embodiments, the low dose of Tacrolimus is about 0.9 times, about 0.8 times, about 0.7 times, about 0.6 times, about 0.5 times, about 0.4 times, about 0.3 times, about 0.2 times, about 0.1 times, about 0.09 times, about 0.08 times, about 0.07 times, about 0.06 times, about 0.05 times, about 0.04 times, about 0.03 times, about 0.02 times, about 0.01 times, about 0.009 times, about 0.08 times or about 0.07 times less than the typical amount used for a particular situation (i.e., typical immunosuppression amounts may differ depending on, for example, the type at organ transplant). In specific embodiments, the low dose of Tacrolimus is about 0.05 mg/kg to about 0.5 mg/kg, more specifically. about 0.05 mg/kg to 0.5 mg/kg, about 0.05 mg/kg to about 0.45 mg/kg, about 0.05 mg/kg to about 0.4 mg/kg, about 0.05 mg/kg to about 0.35 mg/kg, about 0.06 mg/kg to about 0.45 mg/kg, about 0.07 mg/kg to about 0.4 mg/kg, about 0.08 mg/kg to about 0.35 mg/kg, about 0.09 mg/kg to about 0.3 mg/kg, about 0.1 mg/kg to about 0.25 mg/kg, and so on. In a specific embodiment, the low close of Tacrolimus is about 0.05 mg/kg to 0.074 mg/kg.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which Tacrolimus is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount at Tacrolimus together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intraeolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising a low dose of Tacrolimus may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising a low dose of Tacrolimus in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In the case of injections, it is usually convenient to give Tacrolimus by an intravenous route in an amount of about 0.05 mg/kg to about 0.5 mg/kg of Tacrolimus, more specifically, about 0.05 mg/kg to 0.5 mg/kg, about 0.06 mg/kg to about 0.45 mg/kg, about 0.07 mg/kg to about 0.4 mg/kg, about 0.08 mg/kg to about 0.35 mg/kg, about 0.09 mg/kg to about 0.3 mg/kg, about 0.1 mg/kg to about 0.25 mg/kg, and so on.

Doses of a pharmaceutical composition of the present invention can optionally include about 0.05 mg/kg to about 0.5 mg/kg of Tacrolimus including, but not limited to, 0.05, 0.06, 0.07, 0.08. 0.09, 0.1, 0.2, 0.3. 0.4, and/or 0.5 mg/kg/administration or any range, value or fraction thereof, or to achieve a blood level of about 2.0, 2.5, 2.9, 3.0, 3.5, 3,9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9. 20, 12.5, 12.9, 13.0, 13.5, 13.9, 14.0, 14.5, 4.9, 5.0, 5.5, 5.9, 6.0. 6.5, 6.9, 7.0, 7.5. 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20 ng/ml.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day tor about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

The pharmaceutical compositions may further be combined with one or more additional therapeutic agents. The determination of the identity and amount of the pharmaceutical compositions for use in the methods of the present invention can be readily made by ordinarily skilled medical practitioners using standard techniques known in the art. In other specific embodiments, a low dose of Tacrolimus can be administered in combination with an effective amount of another stem cell mobilizer, another immunosuppressive agent, or another therapeutic agent.

Thus, in one aspect, a low dose of Tacrolimus is administered in combination with another stem cell mobilizer. In particular embodiments, the stem cell mobilizer comprises a CXCR4 antagonist. In specific embodiments, the CXCR4 antagonist is TG-0054 (TaiGen Biotechnology Co., Ltd. (Taipei, Taiwan)). In other specific embodiments, the CXCR4 antagonist is AMD3465. In yet other embodiments, the CXCR4 antagonist is AMD3100. AMD3100 (1,1′-[1,4-phenylenebis(methylene)]bis-1,4,8,11-tetraazacyclo-tetradecane) is a symmetric bicyclam, prototype non-peptide antagonist of the CXCR4 chemokine receptor. See U.S. Pat. No. 6,835,731 and No. 6,825,351. The term “AMD” or “AMD3100” is used interchangeably with Plerixafor, rINN, USAN, JM3100, and its trade name, Mozobil™.

In particular embodiments, Tacrolimus and the other stem cell mobilizer (e.g., AMD3100) are administered separately over a period of time following transplantation and/or injury. For example, the treatment regimen for a liver transplant recipient may comprise the following: AMD3100 (1 mg/kg) and Tacrolimus (0.1 mg/kg) at Day 0, 1, 2, 3 and 7 (subcutaneous injection). The treatment regimen may alternatively comprise the following: AMD3100 (1 mg/kg) and Tacrolimus (0.1 mg/kg) at Day 0, 1, 2, 3, 7, 10 and 15 (subcutaneous injection).

As a non-limiting example in kidney transplantation, the treatment regimen may comprise AMD3100 (1 mg/kg) after reperfusion and Day 2, 4, 6, and 10 following transplantation, and Tacrolimus (0.05 mg/kg) after reperfusion and Day 1, 2, 3, 4, 5, 6, 7, and 10 following transplantation (subcutaneous injection). In embodiments involving skin transplantation, the treatment regimen may comprise the following: AMD3100 (1 mg/kg) immediately after transplantation and every two days thereafter, and Tacrolimus (0.1 mg/kg) every day following transplantation (subcutaneous injection).

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions. This application incorporates by reference International Patent Application No. PCT/US2010/059877 (Publication No. WO/2011/072216).

### Materials And Methods

Rat Strains and Care. Lewis (RT1¹) rats were purchased from Harlan ague-Dawley (Indianapolis, Ind.) and used at 8-10 weeks of age. Animals were maintained in the specific pathogen-free facility of Johns Hopkins Medical Institutions. Animals were cared for according to NIH guidelines and under a protocol approved by the Johns Hopkins University Animal Care Committee.

Experimental Groups and Animal Treatment. Lewis rats were divided by six groups including control group (treated with same volume of saline) and low dose of FK-506 (0.05 mg, 0.1 mg or 1.0 mg/kg). FK506 was injected subcutaneously. Animals were sacrificed at 3 hours after injection. Spleen and peripheral blood were collected. Blood cell count was performed in animal pathology laboratory at Johns Hopkins.

Flow Cytometry. Single-cell suspensions (5×10⁵) of peripheral blood monocytes were analyzed for lineage negative and CD133 expression. Nonspecific antibody binding was blocked with goat and rat serum (Sigma) for 30 minutes.

### Results

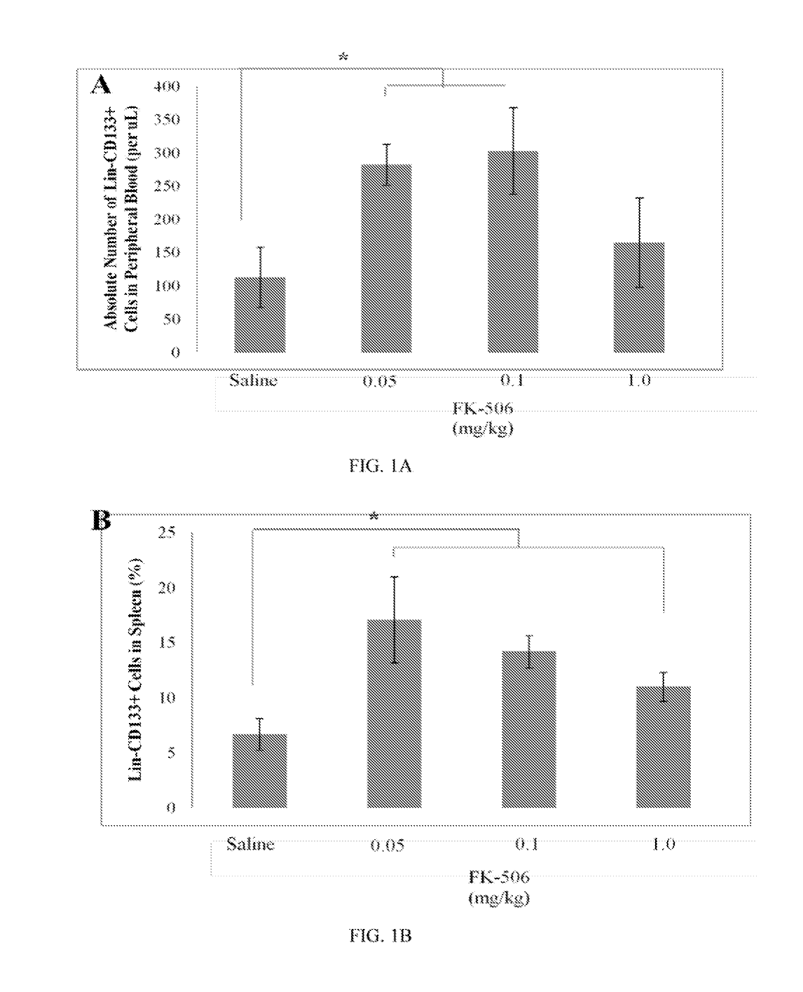

In this study, lineage negative (Lin−) CD133+ stem cell population in peripheral blood and spleen were quantified after FK-506 treatment. Complete blood count (CBC) was measured and stem cell markers were quantified by flow cytometry. The absolute number of stem cells was calculated as WBC (−thous./uL)×% of stem cells. FIG. 1 shows that the absolute number of Lin-CD133+ cells in peripheral blood was significantly increased (about 3 times) in low dose FK-506 (0.05 mg/kg, 0.1 mg/kg) treated animals compare to saline treated animals.

The results described herein will be compared to the effects of either or both of TG-0054 (TaiGen Biotechnology Co., Ltd. (Taipei, Taiwan)) and AMD3100, a commercially available stem cell mobilizing agent (Product No. A5602, Sigma-Aldrich Co, LLC (St. Louis, Mo.)). It is expected that the absolute number of Lin-CD133+ cells in peripheral blood stimulated by low dose FK-506, TG-0054 or AMD3100 will be increased in a statistically significant manner relative to control. A combination of either TG-0054 or AMD3100 and low dose FK-506 (e.g., AMD3100 plus FK-506) is also expected to increase stem cell population in peripheral blood in a statistically significant manner relative to control. In particular, the combination is expected to increase stem cell population in peripheral blood in a statistically significant manner relative to either AMD3100 or low dose FK506 treatment alone.

1. A method of treating an organ transplant recipient comprising administering to the recipient a low dose of Tacrolimus in an amount sufficient to mobilize stem cells to the peripheral blood of the recipient.

2. The method of claim 1, wherein the low dose of Tacrolimus is in the range of about 0.05 mg/kg to about 0.5 mg/kg.

3. The method of claim 1, wherein the low dose of Tacrolimus results in a blood concentration range of about 2 ng/ml to about 20 ng/ml.

4. The method of claim 1, wherein the low dose of Tacrolimus is about 0.05 mg/kg to about 0.1 mg/kg.

5. The method of claim 1, wherein the organ is selected from the group consisting of liver, kidney, skin, heart, lung, intestine, and pancreas.

6. The method of claim 1, wherein the organ is liver.

7. The method of claim 1, wherein the organ is kidney.

8. The method of claim 1, wherein the organ is skin.

9. The method of claim 1, further comprising administering a second agent to mobilize stem cells to the peripheral blood.

10. The method of claim 9, wherein the stem cell mobilizer is selected from the group consisting of AMD3100, AMD3465, TG-0054, G-CSF, GM-CSF, SDF-1, and SCF.

11. The method of claim 9, wherein the stem cell mobilizer is a CXCR4 antagonist.

12. The method of claim 9, wherein the stem cell mobilizer is AMD3100.

13. A method of treating a liver transplant recipient comprising administering Tacrolimus to the recipient in an amount ranging from about 0.05 mg/kg to about 0.5 mg/kg.

14. A method of treating a kidney transplant recipient comprising administering Tacrolimus to the recipient in an amount ranging from about 0.05 mg/kg to about 0.5 mg/kg.

15. A method of treating a skin transplant recipient comprising administering Tacrolimus to the recipient in an amount ranging from about 0.05 mg/kg to about 0.5 mg/kg.

16. A method of treating a patient diagnosed with ischemic injury comprising administering Tacrolimus to the patient in an amount ranging from about 0.05 mg/kg to about 0.5 mg/kg.

17. A method of treating a composite tissue transplant recipient comprising administering Tacrolimus to the recipient in an amount ranging from about 0.05 mg/kg to about 0.5 mg/kg.

18. A method of treating an organ transplant recipient comprising administering a low dose of Tacrolimus to the recipient in an amount sufficient to mobilize CD34⁺ and/or CD133⁺ stem cells to the peripheral blood.

19. The method of claim 18, wherein the low dose of Tacrolimus is about 0.05 mg/kg to about 0.5 mg/kg.

20. A method of treating an organ transplant recipient comprising administering Tacrolimus at a dose of about 0.05 mg/kg to about 0.5 mg/kg to the recipient, wherein the dosage range is sufficient to mobilize CD34⁺ and/or CD133⁺ stem cells to the peripheral blood.

21. A method of treating an organ transplant recipient comprising administering Tacrolimus at a dose of about 0.05 mg/kg to about 0.075 mg/kg to the recipient, wherein the dosage range is sufficient to mobilize CD34⁺ and/or CD133⁺ stem cells to the peripheral blood.
